(19) United States
(12) Patent Application Publication
(10) Pub. No.: US 2014/0161751 A1
(43) Pub. Date: Jun. 12, 2014

(54) STABLE SKIN CARE COMPOSITIONS AND METHODS FOR MANUFACTURING THEM

(71) Applicant: OMP, Inc., Long Beach, CA (US)

(72) Inventors: Jose E. Ramirez, Trumbull, CT (US); Austin McNamara, Villa Park, CA (US); Judy Hattendorf, Marina Del Rey, CA (US); Steve Goldner, Farmington Hills, MI (US)

(21) Appl. No.: 13/942,419

(22) Filed: Jul. 15, 2013

Related U.S. Application Data
(63) Continuation of application No. 11/291,225, filed on Dec. 1, 2005, now abandoned.
(60) Provisional application No. 60/632,440, filed on Dec. 2, 2004.

(51) Int. Cl. A61K 8/02; A61Q 19/02; A61K 8/34

(52) U.S. Cl. CPC A61K 8/0204; A61K 8/347; A61Q 19/02
USPC 424/62

(57) ABSTRACT

Stable hydroquinone compositions useful for skin care have a shelf life of up to three years without discoloration.

### CROSS REFERENCE TO RELATED APPLICATION

This application claims priority benefit of U.S. Provisional Application No. 60/632,440 filed Dec. 2, 2004, the entire disclosure of which is incorporated herein by this reference.

### BACKGROUND

1. Technical Field

The present disclosure relates to stable skin care compositions containing hydroquinone.

2. Background of the Invention

Hydroquinone inhibits the formation of melanin and pigmentation of the skin and, accordingly, has been found to be useful in skin care products, especially products designed to lighten the skin of a user. Unfortunately, hydroquinone compositions turn to a brown color as the hydroquinone in the composition is oxidized. The discoloration of the hydroquinone product is aesthetically undesirable and unacceptable to the consumer. Also, the oxidation by-products of hydroquinone may diminish its effectiveness and irritate the skin of a user.

Although various attempts have been made to solve the problem of discoloration of hydroquinone skin care compositions, applicants are unaware of any stabilized composition that provides the three year shelf-life that is desirable for consumer skin care products. Thus, there remains room for improvement in formulating hydroquinone skin care compositions, especially to provide hydroquinone skin care compositions that do not oxidize and discolor during shipment and storage and which exhibit a three year shelf-life.

### SUMMARY

Hydroquinone compositions are provided herein that are stable for as long as three years at room temperature as shown by accelerated aging tests. In particularly useful embodiments, the present compositions contain hydroquinone, a preservative, a chelating agent, an emulsifier, a humectant, a pH adjuster, an antioxidant, an emollient, a reducing agent, and purified water. In certain embodiments, the present compositions include hydroquinone; one or more preservatives selected from the group consisting of methylparaben, propylparaben, butylparaben and combinations thereof; edetate disodium as a chelating agent; one or more emulsifiers selected from the group consisting of sodium lauryl sulfate, saponins and combinations thereof; glycerin as a humectant; lactic acid as a pH adjuster; one or more antioxidants selected from the group consisting of tocopherol acetate, BHT and combinations thereof; one or more emollients and viscosity building agents selected from the group consisting of cetyl alcohol, stearyl alcohol and combinations thereof; one or more reducing agents selected from the group consisting of ascorbic acid, sodium metabisulfite and combination thereof; and water.

### DETAILED DESCRIPTION OF PREFERRED EMBODIMENTS

The present disclosure relates to stable hydroquinone-containing compositions for skin care. As used herein the term “stable” means that the composition when in a closed container remains within the tolerances and limits set forth in US Pharmacopeia and/or the US FDA guidelines or monographs for compositions containing hydroquinone (or hydroquinone in combination with one or more other active ingredients). The entire US Pharmacopeia and collection of US FDA guidelines or monographs for compositions containing any particular active ingredient or combination of active ingredients are too voluminous to present in their entirety herein and thus are instead incorporated in their entirety by this reference. With respect to topical compositions, the tolerances and limits are frequently presented relative to the labeled amount. With respect to hydroquinone cream, for example, the acceptable tolerance is not less than 94.0 percent and not more than 106.0 percent of the labeled amount of C₆H₆O₂. Those skilled in the art will readily be able to identify the tolerances and limits for other compositions containing hydroquinone.

Stability of the present compositions can be evaluated through accelerated stability studies. In these tests, the packaged composition is maintained at an elevated temperature for a period of time after which it is examined. The exposure to elevated temperatures for a given period correlates to a correspondingly longer period of time at room temperature. Thus, for example, if a product remains within the required tolerances and limits when maintained for a period of 12 weeks at a temperature of 40° C. and 12 months further at room temperature, one can conclude that the product has a shelf life of greater than two and up to three years at room temperature. Those skilled in the art will envision other testing to confirm the stability of the products described herein. Further testing methodology is described in the working examples below.

As those skilled in the art will appreciate, the container-liner-closure system used to store the composition will affect the stability of the active ingredient. It should be understood that a composition need not be stable in all containers to be stable in accordance with this disclosure. Stability in at least one type of container is sufficient for a composition to be stable as that term is used herein.

In particularly useful embodiments, hydroquinone compositions in accordance with the present disclosure can be stable for at least three years at room temperature. Compositions in accordance with some embodiments of this disclosure can be effective in reducing melanin, inhibiting tyrosine, destroying melanocytes, and are thus may be useful as skin lightening compositions.

The hydroquinone compositions of the present disclosure contain hydroquinone and a unique mixture of ingredients and includes an aqueous phase and an oil phase. Hydroquinone is a well-known compound having the general formula:

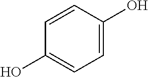

The hydroquinone is present in amounts that provide a benefit to the skin of a user. In embodiments, hydroquinone is present in an amount sufficient to effect depigmentation. Generally, hydroquinone in amounts from about 0.1 to about 10% by weight of the total composition is suitable. In embodiments, hydroquinone is present in an amount from about 1 to about 5% by weight of the total composition. In yet other embodiments, hydroquinone is present in an amount from about 2.5 to about 4.5% by weight of the total composition.

The aqueous phase includes water, humectants, emulsifiers, preservatives, chelating agents, reducing agents and a pH adjuster. Purified water can advantageously be used, such as, for example, de-ionized water or USP water.

Suitable preservatives for the aqueous phase include, but are not limited to methylparaben, sodium butylparaben, benzoic acid and its salts and esters, benzyl alcohol, urea derivatives such as diazolidinyl urea, imidazolidinyl urea, and 2,3-lmidazolidinedione (DMDM hydantoin), sorbic acid and its salts, and the like. Typically, the preservatives are present in an amount from about 0.01 to about 5% by weight of the total composition. In embodiments, the preservatives are present in an amount from about 0.05 to about 1% by weight of the total composition.

Suitable chelating agents for the use in the aqueous phase include, but are not limited to edetate disodium, EDTA (ethylenediaminetetraacetic acid) and its salts, for example, trisodium NTA (nitrilotriacetic acid), etidronic acid and its salts, sodium dihydroxyethylglycinate, citric acid and its salts, and combinations thereof. Typically, the amount of chelating agent(s) is from about 0.01 to about 5% by weight of the total composition. In embodiments, the chelating agents are present in an amount of about 0.05 to about 1% by weight of the total composition.

Suitable humectants for use in the aqueous phase include, but are not limited to polyhydric alcohols including glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, glucose, maltose, sucrose, lactose, xylitose, xylitol, sorbitol, mannitol, maltitol, panthenol, pentaerythritol, and hyaluronic acid and its salts. It should, of course be understood that combinations of two or more humectants can be included in the present compositions. Typically, humectants are present in an amount from about 1 to about 20% by weight of the total composition. In embodiments, humectants are present in an amount from about 2 to about 5% by weight of the total composition.

Suitable emulsifiers for use in the aqueous phase are surfactants. Useful surfactants can be ionic or nonionic, and they can be used alone or in admixture. Illustrative examples of suitable surfactants include cetearyl alcohol and sodium cetearyl sulfate, PEG-1000 monocetyl ether, or quaternary ammonium salts such as alkyl trimethyl ammonium bromide; polyol ester glycerol monostearate and potassium stearate, sodium lauryl sulfate (SLS), and ethoxylated fatty alcohols constitute good coemulsifiers. Fatty acids like stearic acids may be included to regulate the consistency of the emulsion. Optionally, polymers such as carbomers also can be included. Particularly useful emulsifiers for use in the aqueous phase are sodium lauryl sulfate, saponins or combinations thereof. Typically, the emulsifiers are present in an amount from about 1 to about 20% by weight of the total composition. In embodiments, the emulsifiers are present in an amount from about 2 to about 5% by weight of the total composition.

The pH of the aqueous phase can be adjusted to be about 2 to 4, such that the final product has a pH such as between about 2 to 4. Agents suitable for adjusting the pH of the aqueous phase include, but are not limited to citric acid, phosphoric acid, lactic acid or glycolic acid. Typically, the pH adjustment agents are present in an amount from about 0.01 to about 5% by weight of the total composition. In embodiments, the pH adjustment agent is present in an amount from about 0.1 to about 1.0% by weight of the total composition.

Suitable reducing agents for use in the present compositions include, but are not limited to ascorbic acid, propyl gallate and sulfites, including but not limited to sulfites, bisulfites, metabisulfites, their salts, and their derivatives. Sodium metabisulfite is one useful sulfite. Since hydroquinone has a tendency to discolor through oxidation, these reducing agents can be advantageously used because they have greater tendencies to oxidize than hydroquinone. Sodium metabisulfite has the added advantage that it does not discolor by oxidation. In hydroquinone and sodium metabisulfite compositions, it is believed that the sodium metabisulfite oxidizes first and delays the start of any oxidation of the hydroquinone, so that excessive discoloration is delayed or totally avoided. Typically, the reducing agents are present in amounts from about 0.1 to about 10% by weight of the total composition. In embodiments, the reducing agents are present in an amount from about 0.5 to about 5% by weight of the total composition.

The aqueous phase can be prepared by combining the various ingredients while mixing with heating (e.g., to 70-75° C.).

The aqueous phase is mixed with an oil phase. The oil phase can include emollients, preservatives and antioxidants.

Suitable emollients for use in the oil phase include cosmetically acceptable liquid oils. The cosmetically acceptable liquid oil is liquid at room temperature. The cosmetically acceptable liquid oil can be liquid hydrocarbon oil, liquid natural oil, liquid fatty alcohol, liquid fatty acid, liquid fatty acid ester, liquid silicone oil, and paste wax and mixtures thereof.

Non-limiting examples of the liquid hydrocarbons suitable for use in the oil phase include squalane, liquid mineral oil, and liquid polybutene. Non-limiting examples of the liquid natural oil derived from plants useful in the present compositions include almond oil, olive oil, sesame oil, safflower oil, avocado oil, cottonseed oil, jojoba oil, castor oil, soybean oil, palm kernel oil, coconut oil, and hydrogenated vegetable oil. Non-limiting examples of the liquid natural oil derived from animal sources useful in the present compositions include mink oil and egg yolk oil. Non-limiting examples of the liquid fatty alcohol useful in the present compositions are isostearyl alcohol, lanolin alcohol, oleyl alcohol, hexadecyl alcohol, octyldodecanol alcohol, linoleyl alcohol, linolenyl alcohol, lauryl alcohol and arachidyl alcohol. Fatty acid can be natural or synthetic, saturated, unsaturated, linear, or branched. Non-limiting examples of fatty acid useful in the present compositions are caprylic, isostearic, linoleic, ricinoleic, and oleic acid. Non-limiting examples of the liquid fatty acid ester useful in the present compositions are cetyl octanoate, glyceryl trioctanoate, isopropyl linoleate, isopropyl myristate, isopropyl oleate, ethyl laurate, ethyl linoleate, octyl dodecyl myristate, octyl palmitate, octyl isopelargonate, octyl dodecyl lactate, isotridecyl isononanoate, oleyl oleate, isostearyl myristate, neopentyl glycol dioctanoate, and di(capryl/capric acid) propylene glycol and mixtures thereof. Other suitable esters include triglycerides such as caprylic triglycerides, capric triglyceride, isostearic triglyceride and adipic triglyceride. Non-volatile, straight, and branched silicone oils such as dimethicone and phenyl dimethicone are also useful. Other cosmetically acceptable ingredients like sunscreens include octyl methoxy cinnamate, cinoxate, and 2-ethylphexyl p-dimethyaminobenzoate and the like.

Either one kind or two or more kinds of the cosmetically acceptable liquid oil can be used in the present compositions. Particularly useful emollients include cetyl alcohol, stearyl alcohol and combinations thereof. Typically, the emollients are present in an amount from about 5 to about 25% by weight of the total composition. In embodiments, the emollients are present in an amount from about 7.0 to about 15% by weight of the total composition.

Suitable antioxidants for use in the oil phase include, but are not limited to 2,6 ditertiarybutyl-4-methyl phenol (commonly known as butylated hydroxytoluene (BHT)), butylated hydroxyanisole (BHA), tocopherol, tocopheryl acetate, ascorbyl palmitate, propyl gallate, and the like. Typically, the antioxidants are present in an amount from about 0.01 to about 10% by weight of the total composition. In embodiments, the antioxidants are present in an amount from about 0.1 to about 2% by weight of the total composition.

Suitable preservatives for use in the oil phase include propylparaben, isopropylparaben, butylparaben, and isobutylparaben, and the like. The preservatives in the oil phase typically are present in an amount from about 0.01 to about 5% by weight based on the total composition. In embodiments, preservatives are present in an amount from about 0.0.5 to about 2% by weight based in the total composition.

The oil phase can be prepared by simply adding the ingredients for the oil phase into a tank and heating (e.g., to 70-75° C.) with moderate agitation.

The oil phase is then added to the aqueous phase (e.g., at 70-75° C.) with moderate agitation. The present hydroquinone compositions can be prepared under an inert, oxygen-free atmosphere as disclosed in U.S. Pat. No. 4,229,427 the entire disclosure of which is incorporated herein by this reference.

The viscosity of the final hydroquinone composition can be from about 1,000 to about 50,000 centipoise (cps). In embodiments, the viscosity of the final hydroquinone composition is from about 2,500 to about 15,000 cps. The specific gravity of the final composition can be from about 0.5 and 1.5. In embodiments, the specific gravity of the final hydroquinone is from about 0.95 to about 1.05.

In particularly useful embodiments, the final hydroquinone composition may have a substantially white color and be a semi-viscous lotion. In particularly useful embodiments, the present compositions have the ability to substantially maintain its color over time. In such embodiments, the present compositions can appear fresh, elegant and professional for their entire shelf life, ensuring patient or consumer confidence in the product.

Certain embodiments of hydroquinone compositions in accordance with this disclosure have the compositions set forth in Table 1.

TABLE 1

| | | % by weight | Quantity | |
|---|---|---|---|---|
| | | of the total | used to produce | |
| | % of total | composition | 1,500 kg batch | |
| Compound | composition | in Example 1 | in Example 1 | |
| Purified water | 65-85 | 68.32 | 1,025.7 | kilograms |
| Methylparaben | 0.01-0.5 | 0.15 | 2.25 | kilograms |
| Edetate disodium | 0.01-0.5 | 0.30 | 5.0 | kilograms |
| Glycerin | 1-15 | 6.00 | 90.0 | kilograms |
| Sodium lauryl sulfate | 0.01-5 | 1.00 | 15.0 | kilograms |
| Saponins | 0.01-5 | 0.5 | 7.5 | kilograms |
| Lactic Acid 88% | 0.0-5 | 1.0 | 15 | kilograms |
| Cetyl alcohol | 1-25 | 8.00 | 120.0 | kilograms |
| Stearyl alcohol | 0.0-25 | 1.80 | 27.0 | kilograms |
| Propylparaben | 0.01-0.5 | 0.05 | 750.0 | grams |
| Butylparaben | 0.00-0.5 | 0.03 | 450.0 | grams |
| Tocopheryl acetate | 0.01-5 | 0.50 | 7.5 | kilograms |
| BHT | 0.01-0.5 | 0.05 | 750.0 | grams |
| Ascorbic Acid | 0.01-5 | 1.5 | 22.5 | kilograms |
| Sodium metabisulfite | 0.01-5 | 0.7 | 10.5 | kilograms |
| Hydroquinone + 1% | 0.1-10 | 4.04 | 60.6 | kilograms |
| OVR | | | | |
| Octyl | 0.0-8.00 | 3.00 | 45 | kilograms |
| Methoxycinnamate | | | | |
| Oxybenzone | 0.0-8.00 | 3.00 | 45 | kilograms |
| TEA-salicylate | 0.0-5 | | | |
| PPG-2 myristyl ether | 0.0-5 | | | |
| propionate | | | | |
| phenyl trimethicone | 0.0-5 | | | |

The composition can be packaged in suitable containers such as tubes or bottles. Suitable containers are commercially available from a variety of suppliers. A wide variety of containers and suppliers are listed in the CPC Packaging Directory. (See, Buyers' Guide under “Containers” at www.cpcpkg.com).

### Testing Methodology

A stability program was developed to ensure that any product manufactured retained within the specified limits and throughout the period of storage and use, the similar properties and characteristics that it possessed at the time of its manufacture as reported in Table 2 below.

The stability program consisted of accelerated, 9α and long term testing studies. Data collected from the studies determined the shelf life or expiration date of the product. Throughout the study, side or inverted orientation was used for the storage of bottles. The accelerated testing was done by subjecting the product at 40° C.±2° C., 75%±5% relative humidity for one month, two months, and three months. The long term testing was done by subjecting the product at 25° C.±2° C. ambient temperature at intervals of 3, 12, 24, 36, 48 and 60 months. The 9α testing was done by subjecting the product at 40° C. for 3 months, then moving sample to 25° C. for 6 months and then pulling for testing.

Variations of the accelerated testing were chosen for suspensions, topical cream, and ointments. This is called the freeze/thaw challenge test. Products were subjected to 4 (four) cycles. Each cycle entails 6 (six) days at −15° C. and 1 (one) day at controlled room temperature.

Products tested are packaged in the container/closure system in which they are to be marketed. In the case where more than one container/closure system was used for a particular size, testing was done on each container closure system. When the same resin was used for different size container for the same product, only the largest and the smallest size containers were subjected to stability testing for that product. If package 1 (pk1) was of different resin as package 2 (pk2), pk2 was subjected to all tests as pk1.

The pilot batch or the R&D batch were placed on accelerated stability and the first three production batches of a formulation packaged for each container/closure system was placed on full stability testing. Yearly thereafter, one production batch was added to the stability program.

Cosmetic products are subjected to accelerated testing, and retain samples were randomly checked.

The results of various stability studies on a formulation of table 1 in accordance with this disclosure are reported in Table 2 below.

TABLE 2

| | | | | | | | | | | Hydroquinone (4% Label claim) | | | | | |
|---|---|---|---|---|---|---|---|---|---|---|---|---|---|---|---|
| | Appearance | | | | | | | | | Begin | | Middle | | End | |
| | Begin | Middle | End | Viscosity | | | | | | | % of | | % of | | % of |
| | Pass/ | Pass/ | Pass/ | Begin | Mid | End | pH | | | | Label | | Label | | Label |
| Conditions | Fail | Fail | Fail | cps | cps | cps | Begin | Mid | End | Assay | Claim | Assay | Claim | Assay | Claim |
| Initial Bulk | Pass | Pass | Pass | 16,000 | 16,000 | 15,500 | 3.37 | 3.36 | 3.31 | 3.852 | 96.0 | 3.877 | 97.0 | 3.950 | 99.0 |
| 1 Month | Pass | Pass | Pass | 16,000 | 16,250 | 13,500 | 2.90 | 3.05 | 3.18 | 4.110 | 102.8 | 4.115 | 102.9 | 4.118 | 103.0 |
| 40° C., 75% RH | | | | | | | | | | | | | | | |
| 1 Month | Pass | Pass | Pass | 13,500 | 16,000 | 13,500 | 3.05 | 3.13 | 3.17 | 4.074 | 101.9 | 4.117 | 102.9 | 4.096 | 102.4 |
| 30° C., 60% RH | | | | | | | | | | | | | | | |
| 2 Month | Pass | Pass | Pass | 15,500 | 15,000 | 12,500 | 3.01 | 3.15 | 3.14 | 3.905 | 97.6 | 3.884 | 97.1 | 3.947 | 98.7 |
| 40° C., 75% RH | | | | | | | | | | | | | | | |
| 2 Month | Pass | Pass | Pass | 13,000 | 15,000 | 13,250 | 3.01 | 3.09 | 3.10 | 3.883 | 97.1 | 3.888 | 97.2 | 3.809 | 95.2 |
| 30° C., 60% RH | | | | | | | | | | | | | | | |
| 3 Month | Pass | Pass | Pass | 18,000 | 17,250 | 17,000 | 2.84 | 2.41 | 2.49 | 4.026 | 100.7 | 3.972 | 99.3 | 4.022 | 100.6 |
| 40° C., 75% RH | | | | | | | | | | | | | | | |
| 3 Month | Pass | Pass | Pass | 12,500 | 12,500 | 12,500 | 2.76 | 2.75 | 2.84 | 4.047 | 101.2 | 4.049 | 101.2 | 3.980 | 99.5 |
| 30° C., 60% RH | | | | | | | | | | | | | | | |
| 3 Month Bulk | Pass | Pass | Pass | 12,500 | 13,000 | 12,500 | 3.33 | 3.23 | 3.31 | 3.891 | 97.3 | 3.920 | 98.0 | 3.888 | 97.2 |
| 9 Alpha | Pass | Pass | Pass | 17,000 | 16,750 | 16,500 | 2.42 | 2.36 | 2.35 | 3.836 | 96.0 | 3.861 | 96.0 | 3.866 | 97.0 |
| 25° C., 60% RH* | | | | | | | | | | 3.827 | 96.0 | 3.885 | 97.0 | 3.889 | 97.0 |
| 12 Month | Pass | Pass | Pass | 13,000 | 13,250 | 13,500 | 3.15 | 3.21 | 3.09 | 3.921 | 98.0 | 3.904 | 97.6 | 3.917 | 97.9 |
| 25° C., 60% RH* | | | | | | | | | | 3.908 | 97.7 | 3.872 | 97.3 | 3.942 | 98.6 |
| 24 Month | Pass | Pass | Pass | 19,500 | 19,000 | 19,00 | 2.11 | 2.07 | 2.08 | 3.86 | 96.5 | 3.90 | 97.5 | 3.93 | 98.2 |
| 25° C., 60% RH* | | | | | | | | | | 3.86 | 96.5 | 3.91 | 97.8 | 3.91 | 97.8 |
| 3 mos, 40° C., | Pass | Pass | Pass | 20,000 | 20,000 | 20,000 | 2.01 | 2.01 | 2.01 | 3.82 | 95.5 | 3.83 | 9.8 | 3.84 | 96.0 |
| 75% RH Then | | | | | | | | | | 3.84 | 96.0 | 3.85 | 96.2 | 3.82 | 995.5 |
| 25° C., 60% RH | | | | | | | | | | | | | | | |
| for 27 mos* | | | | | | | | | | | | | | | |
| 3 mos, 40° C., | Pass | Pass | Pass | 13,000 | 13,000 | 13,000 | 1.95 | 1.95 | 1.95 | 3.99 | 99.8 | 4.02 | 100.5 | 4.02 | 100.5 |
| 75% RH then | | | | | | | | | | 3.96 | 99.0 | 4.02 | 100.5 | 3.99 | 99.8 |
| 25° C., 60% RH | | | | | | | | | | | | | | | |
| for 28 months* | | | | | | | | | | | | | | | |

*Assay results reported for two sample

### Example I

1025.7 Kg of purified water was added to a premix tank under nitrogen gas. The following water phase ingredients were added under gentle heat to make a 1500 Kg sample. All solids were homogenously dispersed by mixing at about 70-75° C.: 2.25 Kg of methylparaben, 5.0 Kg of edetate disodium, 90 KG glycerin, 15 Kg SLS, 7.5 Kg saponins, 15 Kg lactic acid 88%, 22.5 Kg ascorbic acid, 10.5 Kg sodium metabisulfite, 60.6 Kg hydroquinone (includes 1% overage). The following oil phase ingredients were mixed in a tank at about 70-75° C.: 120 Kg cetyl alcohol, 27 Kg stearyl alcohol, 750 g propylparaben, 450 g butyl paraben, 7.5 Kg tocopheryl acetate, 45 Kg oxybenzone, 45 Kg octyl methoxycinnamate and 750 g BHT. All contents were then transferred under nitrogen gas into a jacketed tank where the oil phase and water phase were mixed and cooled in order to form an emulsion. All solvents were degassed with nitrogen before use and all formulations and intermediate formulations were degassed. The hydroquinone-containing formulation was transferred to storage containers that were purged with nitrogen both before and after filling.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

What is claimed is:

1. A composition comprising:
hydroquinone; a preservative; a chelating agent; an emulsifier; a humectant; a pH adjuster; an antioxidant; an emollient; a reducing agent and water,
wherein the composition maintains an acceptable color for three years.

2. The composition of claim 1, wherein the acceptable color is a color lighter than brown.

3. The composition of claim 1, wherein the composition is prepared in a passivated metal tank, and wherein the discoloration of the composition is slowed compared to the discoloration of the same composition that has not been prepared in the passivated metal tank

4. The composition of claim 1, wherein the composition has a reduced concentration of trace metals, and wherein the reduction in the concentration of trace metals is achieved by passivating a metal tank before preparing the composition in the passivated metal tank.

5. The composition of claim 1, wherein the preservative comprises benzoic acid, benzyl alcohol, butylparaben, diazolidinyl urea, 2,3-Imidazolidinedione, isopropylparaben, isobutylparaben, methylparaben, propylparaben, sodium butylparaben, sorbic acid, or combinations thereof.

6. The composition of claim 1, wherein the chelating agent comprises citric acid, edetate disodium, ethylenediaminetetraacetic acid, etidronic acid sodium dihydroxyethylglycinate, nitrilotriacetic acid, or combinations thereof.

7. The composition of claim 1, wherein the emulsifier comprises cetearyl alcohol, ethoxylated fatty alcohols, PEG-1000 monocetyl ether, alkyl trimethyl ammonium bromide, polyol ester glycerol monostearate, potassium stearate, sodium lauryl sulfate, sodium cetearyl sulfate, saponins, or combinations thereof.

8. The composition of claim 1, wherein the humectant comprises glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, glucose, maltose, sucrose, lactose, xylitose, xylitol, sorbitol, mannitol, maltitol, panthenol, pentaerythritol, hyaluronic acid, or combinations thereof.

9. The composition of claim 1, wherein the pH adjuster comprises citric acid, phosphoric acid, lactic acid, glycolic acid, or combinations thereof.

10. The composition of claim 1, wherein the antioxidant comprises ascorbyl palmitate, 2,6 ditertiarybutyl-4-methyl phenol, butylated hydroxyanisole, tocopherol, tocopheryl acetate, propyl gallate, or combinations thereof.

11. The composition of claim 1, wherein the emollient comprises cetyl alcohol, stearyl alcohol, liquid hydrocarbon oil, liquid natural oil, liquid fatty alcohol, liquid fatty acid, liquid fatty acid ester, liquid silicone oil, paste wax, or combinations thereof.

12. The composition of claim 1, wherein the reducing agent comprises ascorbic acid, propyl gallate, sodium metabisulfite, or combinations thereof.

13. The composition of claim 1, wherein the composition has a viscosity of from about 1,000 to about 50,000 centipoise.

14. The composition of claim 1, wherein the chelating agent is present in the composition in an amount ranging from about 0.05 to about 5%.

15. A composition comprising:
hydroquinone, cetyl alcohol, stearyl alcohol, propylparaben, butylparaben, tocopherol acetate, butylated hydroxytoluene, methylparaben, edetate disodium, glycerin, sodium lauryl sulfate, saponins, lactic acid, and water,
wherein the composition is prepared in a passivated metal tank, and wherein the composition maintains an acceptable color for three years.

16. A composition comprising:
hydroquinone;
one or more preservatives selected from the group consisting of methylparaben, propylparaben, butylparaben and combinations thereof;
edetate disodium;
one or more emulsifiers selected from the group consisting of sodium lauryl sulfate, saponins and combinations thereof;
glycerin;
lactic acid;
one or more antioxidants selected from the group consisting of tocopherol acetate, butylated hydroxytoluene, and combinations thereof;
one or more emollients selected from the group consisting of cetyl alcohol, stearyl alcohol and combinations thereof;
one or more reducing agents selected from the group consisting of ascorbic acid, sodium metabisulfite and combination thereof; and
water,
wherein the composition is prepared in a passivated metal tank, and wherein the composition maintains an acceptable color for three years.

17. A method of making an oil-in-water emulsion comprising mixing hydroquinone; a preservative; a chelating agent; an emulsifier; a humectant; a pH adjuster; an antioxidant; an emollient; a reducing agent and water,
wherein the oil-in-water emulsion is prepared in a passivated metal tank, and wherein the oil-in-water emulsion maintains an acceptable color for three years.

18. The method of claim 17, wherein the preservative comprises benzoic acid, benzyl alcohol, butylparaben, diazolidinyl urea, 2,3-Imidazolidinedione, isopropylparaben, methylparaben, propylparaben, sodium butylparaben, sorbic acid, or combinations thereof.

19. The method of claim 17, wherein the chelating agent comprises citric acid, edetate disodium, ethylenediaminetetraacetic acid, etidronic acid sodium dihydroxyethylglycinate, nitrilotriacetic acid, or combinations thereof.

20. The method of claim 17, wherein the emulsifier comprises cetearyl alcohol, ethoxylated fatty alcohols, PEG-1000 monocetyl ether, alkyl trimethyl ammonium bromide, polyol ester glycerol monostearate, potassium stearate, sodium lauryl sulfate, sodium cetearyl sulfate, saponins, or combinations thereof.

21. The method of claim 17, wherein the humectant comprises glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, glucose, maltose, sucrose, lactose, xylitose, xylitol, sorbitol, mannitol, maltitol, panthenol, pentaerythritol, hyaluronic acid, or combinations thereof.

22. The method of claim 17, wherein the pH adjuster comprises citric acid, phosphoric acid, lactic acid, glycolic acid, or combinations thereof.

23. The method of claim 17, wherein the antioxidant comprises ascorbyl palmitate, 2,6 ditertiarybutyl-4-methyl phenol, butylated hydroxyanisole, tocopherol, tocopheryl acetate, propyl gallate, or combinations thereof.

24. The method of claim 17, wherein the emollient comprises cetyl alcohol, stearyl alcohol, liquid hydrocarbon oil, liquid natural oil, liquid fatty alcohol, liquid fatty acid, liquid fatty acid ester, liquid silicone oil, paste wax, or combinations thereof.

25. The method of claim 17, wherein the reducing agent comprises ascorbic acid, propyl gallate, sodium metabisulfite, or combination thereof.

26. A composition manufactured according to the method of claim 17.
